(19) United States
(12) Patent Application Publication
(10) Pub. No.: US 2026/0088165 A1
(43) Pub. Date: Mar. 26, 2026

(54) METHOD OF REMOTE ACCESS TO ULTRASOUND IMAGING APPARATUS

(71) Applicant: Samsung Medison Co., Ltd., Hongcheon-gun (KR)

(72) Inventors: Jihye Seo, Seoul (KR); Sejae Goh, Seoul (KR); Daehwan Kim, Seoul (KR); Jongrip Lee, Seoul (KR)

(73) Assignee: SAMSUNG MEDISON CO., LTD., Hongcheon-gun (KR)

(21) Appl. No.: 18/989,289

(22) Filed: Dec. 20, 2024

(30) Foreign Application Priority Data
Sep. 24, 2024 (KR) 10-2024-0129432

(51) Int. Cl. G16H 40/67; A61B 8/00; G06Q 30/04; G16H 30/40; G16H 80/00; H04W 12/082

(52) U.S. Cl. CPC G16H 40/67; A61B 8/565; G06Q 30/04; G16H 30/40; G16H 80/00; H04W 12/082

(57) ABSTRACT

Provided is a method of remotely accessing an ultrasound imaging apparatus. The method includes starting an ultrasonic examination by the ultrasound imaging apparatus, activating remote access in response to the starting of the ultrasonic examination, terminating the ultrasonic examination by the ultrasound imaging apparatus, and terminating the activated remote access in response to the terminating of the ultrasonic examination.

### CROSS-REFERENCE TO RELATED APPLICATION

This application is based on and claims priority under 35 U.S.C. § 119 to Korean Patent Application No. 10-2024-0129432, filed on Sep. 24, 2024, in the Korean Intellectual Property Office, the disclosure of which is incorporated by reference herein in its entirety.

### BACKGROUND

### 1. Field

The disclosure relates to a method of remotely accessing an ultrasound imaging apparatus. More specifically, the disclosure relates to technology of providing a one-time remote access when performing ultrasonic remote diagnosis by using an ultrasonic imaging apparatus.

### 2. Description of the Related Art

In recent medical fields, various medical imaging apparatuses for imaging information about tissues of a human body for the purpose of early diagnosis of various diseases or surgery are being widely used. Representative examples of such medical imaging apparatuses may include an ultrasound imaging apparatus, a computed tomography (CT) apparatus, and a magnetic resonance imaging (MRI) apparatus.

Ultrasound imaging apparatuses transmit an ultrasound signal generated by a transducer of a probe to an object and receive information regarding a signal reflected by the object, thereby non-invasively obtaining at least one image of a part (e.g., a soft tissue or a blood stream) inside the object. Ultrasound imaging apparatuses may be used for medical purposes, such as observation of the inside of an object, detection of foreign substances inside the object, and diagnosis of injuries. Such ultrasound imaging apparatuses have various advantages, including stability, real-time display, and safety because there is no exposure to radiation, compared to X-ray imaging apparatuses, and thus, the ultrasound imaging apparatuses are commonly used together with other imaging apparatuses.

A medical professional or professional sonographer, who is a user of an ultrasound imaging apparatus, can perform remote ultrasound diagnosis by remotely accessing an ultrasound imaging apparatus. Existing remote access methods provide remote accessors with full use authority of the ultrasound imaging apparatus. Remote accessors according to existing remote access methods can access ultrasound imaging apparatuses without time restrictions.

However, existing remote access methods have not made it easy to provide one-time remote access authority. Accordingly, existing remote access methods have to require a medical professional or professional sonographer on-site where an ultrasound imaging apparatus exists, to directly control and terminate remote access after using the ultrasound imaging apparatus.

In addition, existing remote access methods unnecessarily provide remote accessors with overly broad use authority. For example, existing remote access methods provide non-medical personnel with unnecessary control authorities even when such personnel access an ultrasound imaging apparatus remotely.

Moreover, existing remote access methods have complicated remote access procedures. Existing remote access methods enable access to a remote access streaming display only when a complicated qualification process is performed. Existing remote access methods are not suitable for one-time remote access for one-time sharing of ultrasound images.

### SUMMARY

Additional aspects will be set forth in part in the description which follows and, in part, will be apparent from the description, or may be learned by practice of the presented embodiments of the disclosure.

A method of remotely accessing an ultrasound imaging apparatus according to an embodiment may include starting an ultrasonic examination by the ultrasound imaging apparatus, activating remote access in response to the starting of the ultrasonic examination, terminating the ultrasonic examination by the ultrasound imaging apparatus, and terminating the activated remote access in response to the terminating of the ultrasonic examination.

A method of remotely accessing an ultrasound imaging apparatus according to an embodiment may include starting an ultrasonic examination by the ultrasound imaging apparatus, and generating a remote access path for activating remote access, in response to the starting of the ultrasonic examination.

A method of remotely accessing an ultrasound imaging apparatus according to an embodiment may include terminating an ultrasonic examination currently being conducted, by the ultrasound imaging apparatus, and terminating the activated remote access by revoking remote access authority granted to a remote accessor, in response to the terminating of the ultrasonic examination.

### BRIEF DESCRIPTION OF THE DRAWINGS

This disclosure may be readily understood by reference to the following detailed description and the accompanying drawings, in which reference numerals refer to structural elements.

The above and other aspects, features, and advantages of certain embodiments of the inventive concept will be more apparent from the following description taken in conjunction with the accompanying drawings, in which:

FIGS. 1A and 1B are block diagrams of an ultrasound imaging system according to an embodiment;

FIGS. 2A, 2B, 2C, and 2D are views illustrating an ultrasound imaging system according to an embodiment;

FIG. 3 is a drawing showing medical personnel remotely accessing an ultrasound imaging apparatus;

FIG. 4 is a diagram showing a remote accessor remotely accessing an ultrasonic imaging apparatus according to an embodiment;

FIG. 5 is a diagram showing activation of remote access to the ultrasonic imaging apparatus according to an embodiment;

FIG. 6 is a diagram showing termination of remote access to the ultrasonic imaging apparatus according to an embodiment;

FIG. 7 is a flowchart of a method of remotely accessing the ultrasonic imaging apparatus according to an embodiment;

FIG. 8 is a flowchart of a method, performed by the ultrasonic imaging apparatus, of activating remote access, according to an embodiment;

FIG. 9 is a flowchart of a method, performed by the ultrasonic imaging apparatus, of activating and terminating remote access, according to an embodiment;

FIG. 10 is a diagram showing storage of a real-time image during remote access to the ultrasonic imaging apparatus according to an embodiment;

FIG. 11 is a diagram showing storage of a past image during remote access to the ultrasonic imaging apparatus according to an embodiment;

FIG. 12 is a diagram showing blocking of abnormal access during remote access to the ultrasonic imaging apparatus according to an embodiment;

FIG. 13 is a diagram showing transmission of invitation information and provision of remote access authority by the ultrasonic imaging apparatus, according to an embodiment;

FIG. 14 is a diagram showing registration of the invitation information and provision of remote access authority by the ultrasonic imaging apparatus, according to an embodiment;

FIG. 15 is a diagram showing provision of a service function during remote access to the ultrasonic imaging apparatus according to an embodiment;

FIG. 16 is a diagram showing charging of a fee for the service function provided during remote access to the ultrasonic imaging apparatus according to an embodiment;

FIG. 17 is a diagram showing transmission of invitation information based on patient information, by the ultrasonic imaging apparatus, and provision of remote access, according to an embodiment;

FIG. 18 is a diagram showing transmission of invitation information to an external device, provision of remote access authority, and transmission of examination result information, by the ultrasonic imaging apparatus through an application, according to an embodiment;

FIG. 19 is a diagram showing provision of medical information during remote access to the ultrasonic imaging apparatus according to an embodiment;

FIG. 20 is a diagram showing provision of remote access authority, by the ultrasonic imaging apparatus, after execution of authentication, according to an embodiment; and

FIG. 21 is a diagram showing provision of remote access based on consent information by the ultrasonic imaging apparatus, according to an embodiment.

### DETAILED DESCRIPTION

The principle of the disclosure is explained and embodiments are disclosed so that the scope of the disclosure is clarified and one of ordinary skill in the art to which the embodiments pertain may implement the embodiments. Embodiments may have various forms.

Throughout the specification, like reference numerals or characters refer to like elements. In the present specification, all elements of embodiments are not explained, but general matters in the technical field of the disclosure or redundant matters between embodiments will not be described. The term “module” or “unit” used herein may be implemented as one or more of software, hardware, or firmware. According to embodiments, a plurality of “modules” or “units” may be implemented as one element, or one “module” or “unit” may include a plurality of elements.

The singular form of a noun corresponding to an item may include one item or a plurality of items, unless a relevant context clearly dictates otherwise.

Each of expressions “A or B”, “at least one of A and B”, “at least one of A or B”, and “one or more of A and/or B”, “A, B, or C,” “at least one of A, B, and C”, and “at least one of A, B, or C” may include any one of listed items and all of at least one combination of the items.

The term “and/or” used herein includes any and all combinations of one or more of the associated listed items.

The terms such as “first”, “second”, “primarily”, or “secondary” used herein may represent various elements regardless of order and/or importance, and do not limit corresponding elements. These terms may be used for distinguishing one element from another element.

Terms such as ‘front surface’, ‘rear surface’, ‘upper surface’, ‘lower surface’, ‘side surface’, ‘left side’, ‘right side’, ‘upper’, and ‘lower’ used herein are defined based on the drawings, and the shape and location of each component are not limited by these terms.

The terms such as “including,” “having,” and “comprising” used herein are intended to indicate the existence of the features, numbers, steps, actions, components, parts, or combinations thereof mentioned in the disclosure, and are not intended to preclude the possibility that one or more other features, numbers, steps, actions, components, parts, or combinations thereof may exist or may be added.

When a component is said to be “connected to”, “coupled to”, “supported by” or “contact” another component, this may include not only a case where the components are directly connected to, coupled to, supported by, or contact each other, but also a case where the components are indirectly connected to, coupled to, supported by, or contact with each other through a third component.

When a component is said to be located “on” another component, this includes not only a case in which a component is in contact with another component, but also a case in which another component exists between two components.

Ultrasound apparatuses according to various embodiments will now be described in detail with reference to the attached drawings. In the drawings, like elements are denoted by like reference numerals, and a repeated explanation thereof will not be given.

An image used herein may be a medical image captured by a medical imaging apparatus, such as a magnetic resonance imaging (MRI) apparatus, a computed tomography (CT) apparatus, an ultrasound imaging apparatus, or an X-ray imaging apparatus.

Throughout the specification, a term ‘object’ is a thing to be imaged, and may include a human, an animal, or a part of a human or animal. For example, the object may include a part of a body (i.e., an organ), a phantom, or the like.

In the disclosure, an “ultrasound image” refers to an image of an object generated or processed based on ultrasound signals transmitted to the object and reflected therefrom.

Embodiments will now be described in detail with reference to the accompanying drawings.

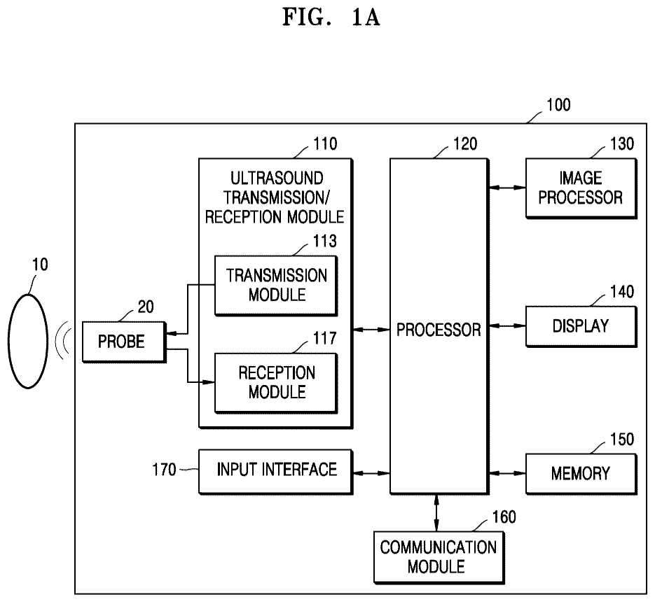

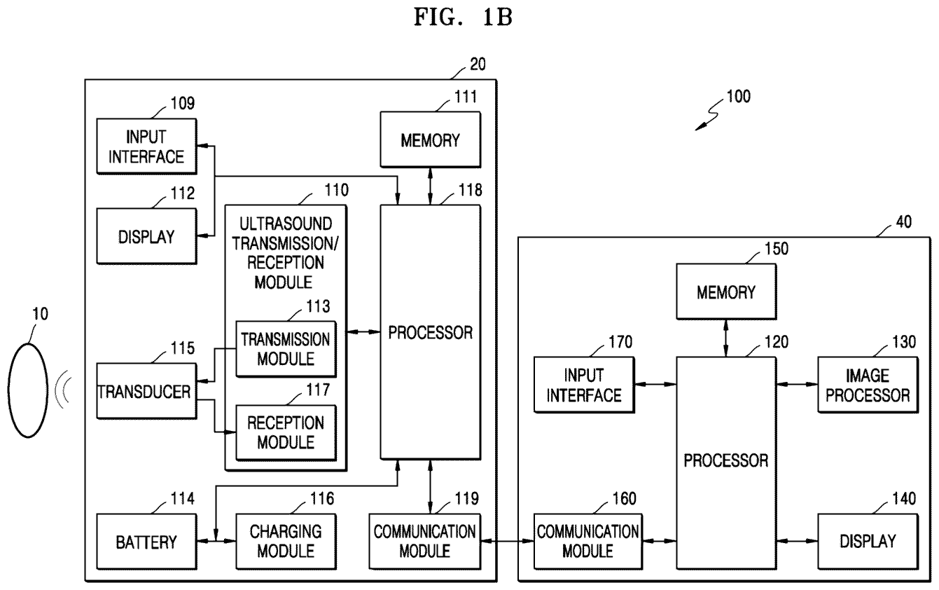

FIGS. 1A and 1B are block diagrams of an ultrasound imaging system 100 according to an embodiment.

Referring to FIGS. 1A and 1B, the ultrasound imaging system 100 may include a probe 20 and an ultrasound imaging apparatus 40.

The ultrasound imaging apparatus 40 may be not only a cart type apparatus, but also a portable apparatus. Examples of portable ultrasound imaging apparatuses may include, but are not limited to, a smartphone, a laptop computer, a personal digital assistant (PDA), and a tablet PC each including a probe and an application. The ultrasound imaging apparatus 40 may also be implemented as an integrated probe.

The probe 20 may include a wired probe that is connected to the ultrasound imaging apparatus 40 by wire to communicate with the ultrasound imaging apparatus 40 by wire, a wireless probe that is wirelessly connected to the ultrasound imaging apparatus 40 to communicate wirelessly with the ultrasound imaging apparatus 40, and/or a hybrid probe that is connected to the ultrasound imaging apparatus 40 by wire or wirelessly to communicate with the ultrasound imaging apparatus 40 by wire or wirelessly.

According to various embodiments, the ultrasound imaging apparatus 40 may include an ultrasound transmission/reception module 110 as shown in FIG. 1A, or the probe 20 may include the ultrasonic transmission/reception module 110 as shown in FIG. 1B. According to various embodiments, the ultrasound imaging apparatus 40 and the probe 20 may include the ultrasound transmission/reception module 110.

According to various embodiments, the probe 20 may further include at least one of an image processor 130, a display 140, or an input interface 170, or a combination thereof. In the disclosure, a description of the ultrasound transmission/reception module 110, the image processor 130, the display 140, or the input interface 170 included in the ultrasound imaging apparatus 40 is also applicable to the ultrasound transmission/reception module 110, the image processor 130, the display 140, or the input interface 170 included in the ultrasound imaging apparatus 40.

FIG. 1A is a block diagram of a configuration of the ultrasound imaging system 100 when the probe 20 is a wired probe or a hybrid probe.

The probe 20 may include a plurality of transducers. The plurality of transducers may be arranged in a predetermined configuration to be implemented as a transducer array. The transducer array may correspond to a one-dimensional (1D) array or a two-dimensional (2D) array. The plurality of transducers may transmit an ultrasound signal to the object 10 according to a transmission signal applied by a transmission module 113. The plurality of transducers may receive an ultrasound signal (echo signal) reflected by the object 10 to form a reception signal. The probe 20 may be integrated with the ultrasound imaging apparatus 40, or may be separate from the ultrasound imaging apparatus 40 and may be connected thereto via a wired communication. The ultrasound imaging apparatus 40 may be connected to one probe 20 or a plurality of probes 210 according to embodiments.

When the probe 20 is a wired probe or a hybrid probe, the probe 20 may include a cable and a connector both connectable to a connector of the ultrasound imaging apparatus 40.

The probe 20 according to an embodiment may be implemented as a 2D probe. When the probe 20 is implemented as a 2D probe, the plurality of transducers included in the probe 20 may be arranged in two dimensions to form a 2D transducer array.

For example, the 2D transducer array may include a plurality of sub-arrays arranged in a second direction different from a first direction and each including a plurality of transducers arranged in the first direction.

When the probe 20 according to an embodiment is implemented as a 2D probe, the ultrasound transmission/reception module 110 may include at least one of an analog beamformer or a digital beamformer. According to an embodiment, the 2D probe may include at least one of an analog beamformer or a digital beamformer, or a combination thereof, according to implementation types.

A processor 120 controls the transmission module 113 to form a transmission signal that is to be applied to each of the plurality of transducers 115, considering respective positions and focal points of the plurality of transducers included in the probe 20.

The processor 120 may control a reception module 115 to generate ultrasonic data by performing analog-to-digital conversion on a reception signal received from the probe 20 and summing a digital reception signal considering the respective positions and focus points of the plurality of transducers.

When the probe 20 is implemented as a 2D probe, the processor 120 may calculate a time delay value for digital beamforming for each sub-array with respect to each of the plurality of sub-arrays included in the 2D transducer array. The processor 120 may calculate a time delay value for analog beamforming with respect to each of the plurality of transducer included in one of the plurality of sub-arrays. The processor 120 may control the analog beamformer and the digital beamformer to form a transmission signal that is to be applied to each of the plurality of transducers, according to the time delay value for analog beamforming and the time delay value for digital beamforming. The processor 120 may control the analog beamformer to sum signals received from the plurality of transducers for each sub-array according to the time delay value for analog beamforming. The processor 120 may control the ultrasound transmission/reception module 110 to perform analog-to-digital conversion with respect to a result of the summation of the signals for each sub-array. The processor 120 may control the digital beamformer to generate ultrasound data, by summing digital signals according to the time delay value for digital beamforming.

The image processor 130 generates or processes an ultrasound image by using the generated ultrasound data.

The display 140 may display the generated ultrasound image, and various pieces of information processed by the ultrasound imaging apparatus 40 or the probe 20. The probe 20 or the ultrasound imaging apparatus 40 may include one display 140 or a plurality of displays 140 according to implementation types. The display 140 may include a touch panel or touch screen. The display 140 may include a flexible display.

The processor 120 may control an overall operation of the ultrasound imaging apparatus 40 and may control operations of the components of the ultrasound imaging apparatus 40. The processor 120 may perform or control various operations or functions of the ultrasound imaging apparatus 40 by executing programs or instructions stored in the memory 150. The processor 120 may receive a control signal from the input interface 170 or an external device to control an operation of the ultrasound imaging apparatus 40.

The ultrasound imaging apparatus 40 may include a communication module 160, and may be connected to and communicate with external devices (e.g., the probe 20, a server, a medical apparatus, and a portable device (smartphone, tablet personal computer (PC), wearable device, etc.), via the communication module 160.

The communication module 160 may include at least one component that enables communication with the external devices. The communication module 160 may include, for example, at least one of a close-distance communication module, a wired communication module, or a wireless communication module.

The communication module 160 may receive a control signal or data from an external device. The processor 120 may control an operation of the ultrasound imaging apparatus 40 according to the control signal received through the communication module 160. By transmitting the control signal to the external device via the communication module 160, the processor 120 may control the external device according to the transmitted control signal. The external device may operate according to the control signal received from the ultrasound imaging apparatus 40, or process data received from the ultrasound imaging apparatus 40.

A program or application related to the ultrasound imaging apparatus 40 may be installed in the external device. The program or application installed in the external device may control the ultrasonic imaging apparatus 40, or may operate according to the control signal or data received from the ultrasonic imaging apparatus 40.

The external device may receive or download the program or application related to the ultrasound imaging apparatus 40 from the ultrasound imaging apparatus 40, the probe 20, or a server, and may provide and execute the program or application in the external device. The ultrasound imaging apparatus 40, the probe 20, or the server providing the program or application may include a recording medium that stores instructions, commands, installation files, executable files, or related data of the program or application. The external device may also be sold with programs or applications provided.

The memory 150 may store various pieces of data or various programs for driving and controlling the ultrasound imaging apparatus 40, input/output ultrasound data, ultrasound images, etc.

The input interface 170 may receive a user input for controlling the ultrasound imaging apparatus 40. For example, the user input may include, but is not limited to, an input for manipulating a button, a key pad, a mouse, a trackball, a jog switch, a knop, or the like, an input for touching a touch pad or a touch screen, a voice input, a motion input, and a biometric data input (e.g., iris recognition and fingerprint recognition).

FIG. 1B is a block diagram showing control by the ultrasound imaging system 100 when the probe 20 is a wired probe or a hybrid probe.

Of course, according to various embodiments, the ultrasound imaging apparatus 40 shown in FIG. 1B may be replaced by the ultrasound imaging apparatus 40 described above with reference to FIG. 1A.

Of course, according to various embodiments, the probe 20 shown in FIG. 1A may be replaced by the probe 20 described above with reference to FIG. 1B.

The probe 20 may include a display 112, a transmission module 113, a battery 114, a transducer 115, a charging module 116, a reception module 117, an input interface 109, a processor 118, and a communication module 119. In FIG. 1B, the probe 20 is shown as including both the transmission module 113 and the reception module 117. However, according to implementation types, the probe 20 may include only some of the transmission module 113 and the reception module 117, or some of the transmission module 113 and the reception module 117 may be included in the ultrasound imaging apparatus 40. According to an embodiment, the probe 20 may further include an image processor 130.

The transducer 115 may include a plurality of transducers. The plurality of transducers may be arranged in a predetermined configuration to be implemented as a transducer array. The transducer array may correspond to a one-dimensional (1D) array or a two-dimensional (2D) array. The plurality of transducers may transmit an ultrasound signal to the object 10 according to a transmission signal applied by the transmission module 113. The plurality of transducers may receive an ultrasound signal reflected by the object 10 and may form or generate an electrical reception signal.

The charging module 116 may charge the battery 114. The charging module 116 may receive power from an external source. According to an embodiment, the charging module 116 may receive power wirelessly. According to an embodiment, the charging module 116 may receive power by wire. The charging module 116 may transmit the received power to the battery 114.

The processor 118 controls the transmission module 113 to generate or form a transmission signal that is to be applied to each of the plurality of transducers 115, considering respective positions and focal points of the plurality of transducers.

The processor 118 may control the reception module 117 to generate ultrasonic data by performing analog-to-digital conversion on a reception signal received from the transducer 115 and summing a digital reception signal considering the respective positions and focus points of the plurality of transducers. According to an embodiment, when the probe 20 includes the image processor 130, the probe 20 may generate an ultrasound image by using the generated ultrasound data.

When the probe 20 is implemented as a 2D probe, the processor 118 may calculate a time delay value for digital beamforming for each sub-array with respect to each of the plurality of sub-arrays included in the 2D transducer array. The processor 118 may calculate a time delay value for analog beamforming with respect to each of the plurality of transducer included in one of the plurality of sub-arrays. The processor 118 may control the analog beamformer and the digital beamformer to form a transmission signal that is to be applied to each of the plurality of transducers, according to the time delay value for analog beamforming and the time delay value for digital beamforming. The processor 118 may control the analog beamformer to sum signals received from the plurality of transducers for each sub-array according to the time delay value for analog beamforming. The processor 118 may control the ultrasound transmission/reception module 110 to perform analog-to-digital conversion with respect to a result of the summation of the signals for each sub-array. The processor 118 may control the digital beamformer to generate ultrasound data, by summing digital signals according to the time delay value for digital beamforming.

The processor 118 may control an overall operation of the probe 20 and may control operations of the components of the probe 20. The processor 118 may perform or control various operations or functions of the probe 20 by executing programs or instructions stored in the memory 111. The processor 118 may receive a control signal from the input interface 109 of the probe 20 or an external device (e.g., the ultrasound imaging apparatus 40) to control an operation of the probe 20. The processor 118 may receive a control signal from the input interface 109 or the external device to control an operation of the probe 20. The input interface 109 may receive a user input for controlling the probe 20. For example, the user input may include, but is not limited to, an input for manipulating a button, a key pad, a mouse, a trackball, a jog switch, a knop, or the like, an input for touching a touch pad or a touch screen, a voice input, a motion input, and a biometric data input (e.g., iris recognition and fingerprint recognition).

The display 112 displays an ultrasound image generated by the probe 20, an ultrasound image generated by processing ultrasound data generated by the probe 20, an ultrasound image received from the ultrasound imaging apparatus 40, or various pieces of information that are processed by the ultrasound imaging system 100. The display 112 may further display status information of the probe 20. The status information of the probe 20 may include device information of the probe 20, battery status information of the probe 20, frequency band information of the probe 20, output information of the probe 20, information regarding occurrence or non-occurrence of abnormality of the probe 20, setting information of the probe 20, or temperature information of the probe 20.

The probe 20 may include one display 112 or a plurality of displays 112 according to implementation types. The display 112 may include a touch panel or touch screen. The display 112 may include a flexible display.

The communication module 119 may wirelessly transmit the generated ultrasound data or ultrasound image to the ultrasound imaging apparatus 40 through a wireless network. The communication module 119 may receive a control signal or data from the ultrasound imaging apparatus 40.

The ultrasound imaging apparatus 40 may receive the ultrasound data or the ultrasound image from the probe 20.

According to an embodiment, when the probe 20 includes the image processor 130 capable of generating an ultrasound image by using ultrasound data, the probe 20 may transmit the ultrasound image generated by the image processor 130 or the ultrasound data to the ultrasound imaging apparatus 40.

According to an embodiment, when the probe 20 does not include the image processor 130 capable of generating an ultrasound image by using ultrasound data, the probe 20 may transmit the ultrasound data to the ultrasound imaging apparatus 40. The ultrasound data may include ultrasound raw data, and the ultrasound image may refer to ultrasound image data.

The ultrasound imaging apparatus 40 may include a processor 120, an image processor 130, a display 140, a memory 150, a communication module 160, and an input interface 170.

The image processor 130 generates or processes the ultrasound image by using the ultrasound data received from the probe 20.

The display 140 may display the ultrasound image received from the probe 20, the ultrasound image generated by processing the ultrasound data received from the probe 20, or various pieces of information that are processed by the ultrasound imaging system 100. The ultrasound imaging apparatus 40 may include one display 140 or a plurality of displays 140 according to implementation types. The display 140 may include a touch panel or touch screen. The display 140 may include a flexible display.

The processor 120 may control an overall operation of the ultrasound imaging apparatus 40 and may control operations of the components of the ultrasound imaging apparatus 40. The processor 120 may perform or control various operations or functions of the ultrasound imaging apparatus 40 by executing programs or instructions stored in the memory 150. The processor 120 may receive a control signal from the input interface 170 or an external device to control an operation of the ultrasound imaging apparatus 40.

The ultrasound imaging apparatus 40 may include a communication module 160, and may be connected to and communicate with external devices (e.g., the probe 20, a server, a medical apparatus, and a portable device (smartphone, tablet personal computer (PC), wearable device, etc.), via the communication module 160.

The communication module 160 may include at least one component that enables communication with the external devices. The communication module 160 may include, for example, at least one of a close-distance communication module, a wired communication module, or a wireless communication module.

The communication module 160 of the ultrasonic imaging apparatus 40 and the communication module 119 of the probe 20 may communicate using a network or using a short-range wireless communication method. For example, the communication module 160 of the ultrasonic imaging apparatus 40 and the communication module 119 of the probe 20 may communicate with each other by using one of wireless data communication methods including, for example, a wireless LAN, Wi-Fi, Bluetooth, Zigbee, Wi-Fi Direct (WFD), infrared communication (IrDA), Bluetooth Low Energy (BLE), Near Field Communication (NFC), Wireless Broadband Internet (Wibro), World Interoperability for Microwave Access (WiMAX), a shared wireless access protocol (SWAP), Wireless Gigabit Alliance (WiGig), RF communication, and 60 GHZ millimeter (mm) wave short-range communication.

To this end, the communication module 160 of the ultrasonic imaging apparatus 40 and the communication module 119 of the probe 20 may include at least one of a wireless LAN communication module, a Wi-Fi communication module, a Bluetooth communication module, a Zigbee communication module, a WFD communication module, an IrDA communication module, a BLE communication module, an NFC communication module, a Wibro communication module, a WiMAX communication module, a SWAP communication module, a WiGig communication module, an RF communication module, or a 60 GHz mm wave short-range communication module.

According to an embodiment, the probe 20 may transmit device information (e.g., ID information) of the probe 20 to the ultrasound imaging apparatus 40 by using a first communication method (e.g., BLE), and may be paired wirelessly with the ultrasound imaging apparatus 40. The probe 20 may transmit the ultrasound data and/or the ultrasound image to the paired ultrasound imaging apparatus 40.

The device information of the probe 20 may include various pieces of information related to a serial number, model name, or battery status of the probe 20.

The ultrasound imaging apparatus 40 may receive the device information (e.g., ID information) of the probe 20 from the probe 20 by using the first communication method (e.g., BLE), and may be paired wirelessly with the probe 20. The ultrasound imaging apparatus 40 may transmit an activation signal to the paired probe 20 and may receive the ultrasound data and/or the ultrasound image from the probe 20. In this case, the activation signal may include a signal for controlling an operation of the probe 20.

According to an embodiment, the probe 20 may transmit the device information (e.g., ID information) of the probe 20 to the ultrasound imaging apparatus 40 by using the first communication method (e.g., BLE), and may be paired wirelessly with the ultrasound imaging apparatus 40. The probe 20 may transmit the ultrasound data and/or the ultrasound image to the ultrasound imaging apparatus 40 paired with the probe 20 by using the first communication method, by using a second communication method (e.g., 60 GHz mm waves or Wi-Fi).

The ultrasound imaging apparatus 40 may receive the device information (e.g., ID information) of the probe 20 from the probe 20 by using the first communication method (e.g., BLE), and may be paired wirelessly with the probe 20. The ultrasound imaging apparatus 40 may transmit an activation signal to the paired probe 20, and may receive the ultrasound data and/or the ultrasound image from the probe 20 by using the second communication method (e.g., 60 GHZ mm waves or Wi-Fi).

According to an embodiment, the first communication method used to pair the probe 20 with the ultrasound imaging apparatus 40 may have a lower frequency band than a frequency band of the second communication method used by the probe 20 to transmit the ultrasound data and/or the ultrasound image to the ultrasound imaging apparatus 40.

The display 140 of the ultrasound imaging apparatus 40 may display user interfaces (UIs) indicating the device information of the probe 20. For example, the display 140 may display a UI or the like indicating identification information of the probe 20, a pairing method indicating a pairing method with the probe 20, a data communication status between the probe 20 and the ultrasound imaging apparatus 40, a method of performing data communication with the ultrasound imaging apparatus 40, or the battery status of the probe 20.

When the probe 20 includes the display 112, the display 112 of the probe 20 may display a UI indicating the device information of the probe 20. For example, the display 112 may display a UI or the like indicating identification information of the probe 20, a pairing method indicating a pairing method with the probe 20, a data communication status between the probe 20 and the ultrasound imaging apparatus 40, a method of performing data communication with the ultrasound imaging apparatus 40, or the battery status of the probe 20.

The communication module 160 may receive a control signal or data from an external device. The processor 120 may control an operation of the ultrasound imaging apparatus 40 according to the control signal received through the communication module 160.

By transmitting the control signal to the external device via the communication module 160, the processor 120 may control the external device according to the transmitted control signal. The external device may operate according to the control signal received from the ultrasound imaging apparatus 40, or process data received from the ultrasound imaging apparatus 40.

The external device may receive or download the program or application related to the ultrasound imaging apparatus 40 from the ultrasound imaging apparatus 40, the probe 20, or a server, and may provide and execute the program or application in the external device. The ultrasound imaging apparatus 40, the probe 20, or the server providing the program or application may include a recording medium that stores instructions, commands, installation files, executable files, or related data of the program or application. The external device may also be sold with programs or applications provided.

The memory 150 may store various pieces of data or various programs for driving and controlling the ultrasound imaging apparatus 40, input/output ultrasound data, ultrasound images, etc.

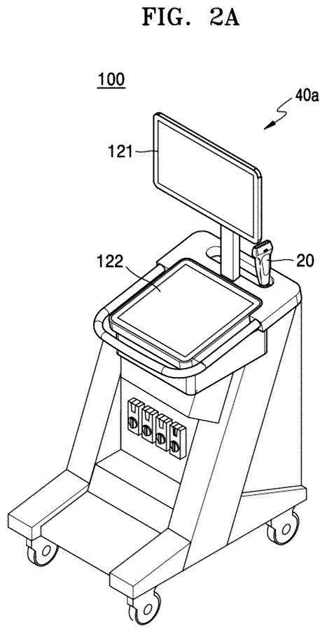

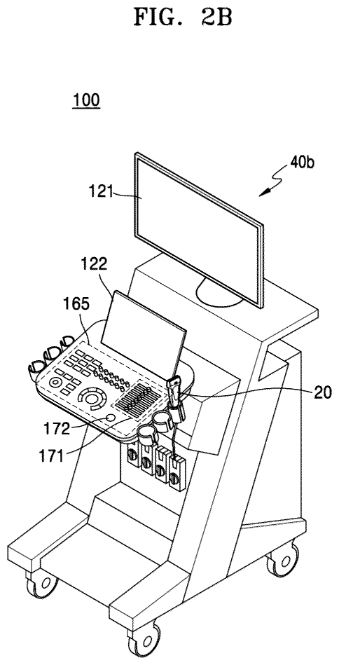

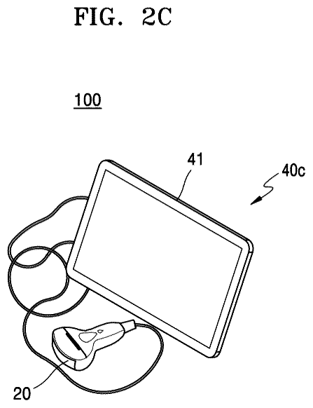

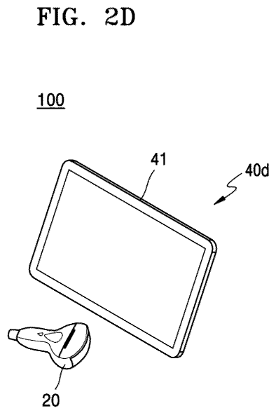

An example of the ultrasound imaging system 100 according to an embodiment will now be described with reference to FIGS. 2A, 2B, 2C, and 2D.

FIGS. 2A, 2B, 2C, and 2D are views illustrating ultrasound imaging apparatuses according to an embodiment.

Referring to FIGS. 2A and 2B, ultrasound imaging apparatuses 40a and 40b may include a main display 121 and a sub-display 122. The main display 121 and the sub-display 122 may correspond to the display 140 of FIGS. 1A and 1B. At least one of the main display 121 or the sub-display 122 may be implemented using a touch screen. At least one of the main display 121 or the sub-display 122 may display various pieces of information that are processed by the ultrasound imaging apparatuses 40a and 40b, or an ultrasound image. At least one of the main display 121 or the sub-display 122 may be implemented using a touch screen, and may provide a graphical user interface (GUI) to thereby receive data for controlling the ultrasound imaging apparatuses 40a and 40b from a user. For example, the main display 121 may display an ultrasound image, and the sub-display 122 may display a control panel for controlling display of the ultrasound image, in a GUI form. The sub-display 122 may receive data for controlling display of an image, via a control panel displayed in a GUI form. For example, a Time Gain Compensation (TGC) button, a Lateral Gain Compensation (LGC) button, a Freeze button, a trackball, a jog switch, or a knob may be provided as a GUI on the sub-display 122.

The ultrasound imaging apparatuses 40a and 40b may control display of an ultrasound image displayed on the main display 121, by using the received control data. The ultrasound imaging apparatuses 40a and 40b may be connected to the probe 20 by wire or wirelessly to transmit and receive ultrasound signals to an object.

Referring to FIG. 2B, the ultrasound imaging apparatus 40b may further include a control panel 165 in addition to the main display 121 and the sub-display 122. The control panel 165 may include buttons, a trackball, a jog switch, a knob, etc., and may receive data for controlling the ultrasonic imaging apparatus 40b from the user. For example, the control panel 165 may include a TGC button 171, a Freeze button 172, etc. The TGC button 171 is a button for setting a TGC value for each depth of an ultrasound image. When an input of the Freeze button 172 is detected while an ultrasound image is being scanned, the ultrasound imaging apparatus 40b may maintain the display of a frame image at that point of time, capture the frame image at that point of time, or store the frame image at that point of time.

The buttons, the trackball, the jog switch, the knob, etc. included in the control panel 165 may be provided as a GUI on the main display 121 or sub-display 122. The ultrasound imaging apparatuses 40a and 40b may be connected to the probe 20 to transmit and receive ultrasound signals to an object.

The ultrasonic imaging apparatuses 40a and 40b may include various types of input/output interfaces such as a speaker, an LED, and a vibration device. For example, the ultrasonic imaging apparatuses 40a and 40b may output various pieces of information in the form of graphics, sound, or vibration through an input/output interface. The ultrasound imaging apparatuses 40a and 40b may output various notifications or data through input/output interfaces.

Referring to FIGS. 2C and 2D, ultrasound imaging apparatuses 40c and 40d may be implemented as portable devices. Examples of the ultrasound imaging apparatuses 40c and 40d may include, but are not limited to, a smartphone, a laptop computer, a personal digital assistant (PDA), or a tablet PC each including a probe and an application.

The ultrasound imaging apparatus 40c may include a main body 41. Referring to FIG. 2C, the probe 20 may be connected to one side of the main body 41 by wire. To this end, the main body 41 may include a connection terminal from which a cable connected to the probe 20 is detachable. The probe 20 may include a cable including a connection terminal connectable to the main body 41.

Referring to FIG. 2D, the probe 20 may be wirelessly connected to the ultrasound imaging apparatus 40d. The main body 41 may include an input/output interface (e.g., a touch screen). The input/output interface may display, for example, ultrasound images, various pieces of information processed by an ultrasound imaging apparatus, or a GUI.

The ultrasound imaging apparatus 40d and the probe 20 may establish communication or be paired, according to short-range wireless communication. For example, the ultrasound imaging apparatus 40d and the probe 20 may perform communication by using Bluetooth, BLE, Wi-Fi, Wi-Fi Direct, or the like.

The ultrasound imaging apparatuses 40c and 40d may execute a program or application related to the probe 20 to control the probe 20 and output information related to the probe 20. The ultrasound imaging apparatuses 40c and 40d may perform an operation related to the probe 20 while communicating with a certain server. The probe 20 may be registered in the ultrasound imaging apparatuses 40c and 40d or may be registered in a certain server. The ultrasound imaging apparatuses 40c and 40d may communicate with the registered probe 20 and may perform an operation related to the probe 20.

The ultrasonic imaging apparatuses 40c and 40d may include various types of input/output interfaces such as a speaker, an LED, and a vibration device. For example, the ultrasonic imaging apparatuses 40c and 40d may output various pieces of information in the form of graphics, sound, or vibration through an input/output interface. The ultrasound imaging apparatuses 40c and 40d may output various notifications or data through input/output interfaces.

According to an embodiment, the ultrasound imaging apparatus 40a, 40b, 40c, or 40d may process an ultrasound images or obtain additional information from the ultrasound image, by using an artificial intelligence (AI) model. According to an embodiment, the ultrasound imaging apparatus 40a, 40b, 40c, or 40d may generate an ultrasound image or perform correction, image quality improvement, encoding, decoding, or the like on the ultrasound image, by using an AI model. According to an embodiment, the ultrasound imaging apparatus 40a, 40b, 40c, or 40d may perform baseline definition, anatomical information obtainment, lesion information obtainment, surface extraction, boundary definition, length measurement, area measurement, volume measurement, annotation creation, or the like from an ultrasound image, by using an AI model.

The AI model may be provided on the ultrasound imaging apparatus 40a, 40b, 40c, or 40d, or may be provided on a server.

The AI model may be implemented using various artificial neural network models or deep neural network models. The AI model may be trained and created using various machine learning algorithms or deep learning algorithms. The AI model may be implemented using a model such as a Convolutional Neural Network (CNN), a Recurrent Neural Network (RNN), a Generative Adversarial Network (GAN), or a Long Short-Term Memory (LSTM).

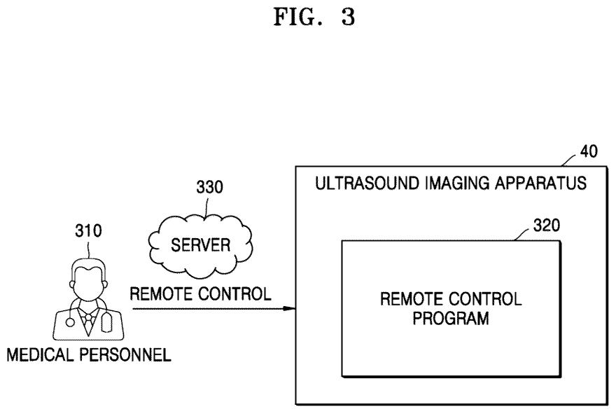

FIG. 3 is a drawing showing a medical personnel 310 remotely accessing the ultrasound imaging apparatus 40.

The ultrasound imaging apparatus 40 may be remotely accessed by the medical personnel 310. The medical personnel 310 may remotely access the ultrasound imaging apparatus 40 and remotely control the ultrasound imaging apparatus 40.

The ultrasound imaging apparatus 40 may include a remote control program 320. The remote control program 320 may receive a control command from the medical personnel 310 remotely connected thereto. The remote control program 320 may control the ultrasonic imaging apparatus 40 so that the ultrasonic imaging apparatus 40 may operate according to the control command.

The ultrasound imaging apparatus 40 may receive the control command from the medical personnel 310 via a server 330. The server 330 may support an environment where the medical personnel 310 is able to remotely access the ultrasound imaging apparatus 40. For example, the server 330 may be a control program for the ultrasonic imaging apparatus 40 that supports a remote access function. For example, the server 330 may be a control program, such as SonoSync™.

A conventional method of remote access to the ultrasound imaging apparatus 40 provides unrestricted remote control authority to a remote accessor, such as the medical personnel 310. The conventional method of remote access to the ultrasonic imaging apparatus 40 enables remote access to the ultrasonic imaging apparatus 40 without time limitations. The conventional method of remote access to the ultrasonic imaging apparatus 40 is a remote access method suitable for the medical personnel 310 or professional sonographers as the medical personnel 310 or professional sonographers could control the ultrasound imaging apparatus 40 while maintaining the connection.

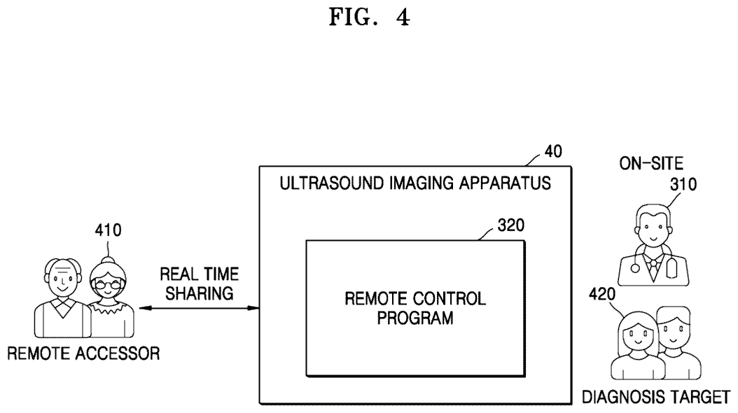

FIG. 4 is a diagram showing a remote accessor 410 remotely accessing the ultrasonic imaging apparatus 40 according to an embodiment.

The medical personnel 310 may use the ultrasound imaging apparatus 40 on site. The remote accessor 410 may remotely access the ultrasound imaging apparatus 40. The remote accessor 410 may share, in real time, the ultrasound image obtained by the ultrasound diagnosis apparatus 40. The remote accessor 410 may see an ultrasound image of a diagnosis target 420 obtained by the ultrasound diagnosis apparatus 40.

Needs for one-time access by non-medical personnel may arise. For example, there may be a case where non-medical persons (e.g., parents' relatives and parents-in-law) in remote locations want to view an ultrasound image of a fetus acquired by the ultrasound imaging apparatus 40 in real time. As another example, there may be a case where the medical personnel 310 wants to easily share an ultrasound image with an external consultant in a remote location.

In a one-time access method of a non-medical personnel according to the disclosure, limited remote access authority may be provided to the remote accessor 410. In the one-time access method of a non-medical personnel according to the disclosure, remote connection may be possible only during a time when an ultrasound image is obtained by the ultrasound imaging apparatus 40 and shared. The one-time access method of non-medical personnel according to the disclosure may be a remote access method suitable for non-medical personnel because the remote accessor 410 may be provided with only limited authority to view the ultrasound image obtained by the ultrasound imaging apparatus 40 excluding the authority to control the ultrasound imaging apparatus 40.

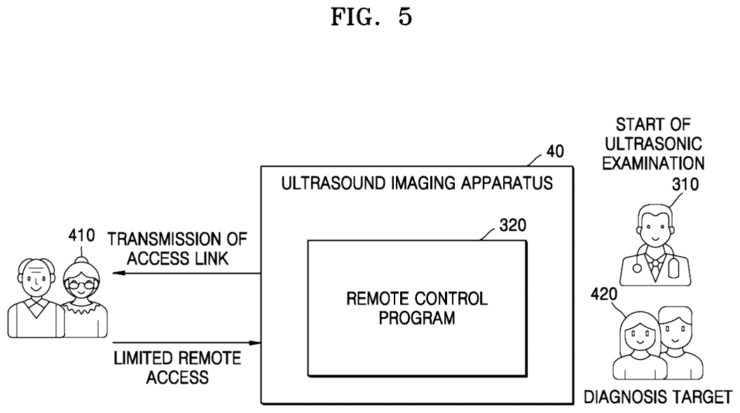

FIG. 5 is a diagram showing activation of remote access to the ultrasonic imaging apparatus 40 according to an embodiment.

The ultrasound imaging apparatus 40 may start an ultrasonic examination. The ultrasound imaging apparatus 40 may start an ultrasonic examination of the diagnosis target 420 under a control by the medical personnel 310. The ultrasonic imaging apparatus 40 may provide a temporary remote access method to the remote accessor 410 in connection with an ultrasonic examination. The ultrasonic imaging apparatus 40 may provide a limited remote access authority to the remote accessor 410 in connection with an ultrasonic examination.

The ultrasound imaging apparatus 40 may activate an remote access in response to the start of the ultrasonic examination. The ultrasound imaging apparatus 40 may generate a remote access path for activating remote access in response to the start of the ultrasonic examination. The ultrasound imaging apparatus 40 may transmit an access link including the remote access path to the remote accessor 410 in response to the start of the ultrasonic examination. The ultrasound imaging apparatus 40 may transmit, to the remote accessor 410, an access link capable of remotely accessing the ultrasound imaging apparatus 40.

The ultrasonic imaging apparatus 40 may transmit the access link to an external device of the remote accessor 410 when the ultrasonic examination starts. For example, the ultrasound imaging apparatus 40 may transmit an invitation request in an e-mail or QR code form to the external device of the remote accessor 410 when the ultrasonic examination starts. The external device of the remote accessor 410 may include a display capable of displaying an ultrasound image, such as a terminal device carried by the remote accessor 410, a PC of the remote accessor 410, or a tablet of the remote accessor 410

The remote accessor 410 may limitedly and remotely access the ultrasound imaging apparatus 40 by using the access link. The ultrasonic imaging apparatus 40 may provide the remote accessor 410 with the ultrasound image obtained by the ultrasound imaging apparatus 40. The remote accessor 410 may view the ultrasound image provided by the ultrasound imaging apparatus 40, by using the external device of the remote accessor 410.

The ultrasound imaging apparatus 40 may grant limited remote access authority to the remote accessor 410. For example, the ultrasound imaging apparatus 40 may provide, to the remote accessor 410, a magnifying function for the ultrasound image provided to the remote accessor 410. For example, the ultrasound imaging apparatus 40 may provide, to the remote accessor 410, a reduction function for the ultrasound image provided to the remote accessor 410. For example, the ultrasound imaging apparatus 40 may provide, to the remote accessor 410, a rotation function for the ultrasound image provided to the remote accessor 410. For example, the ultrasound imaging apparatus 40 may provide, to the remote accessor 410, an adjustment function for a cine bar for adjusting a time point of the ultrasound image provided to the remote accessor 410.

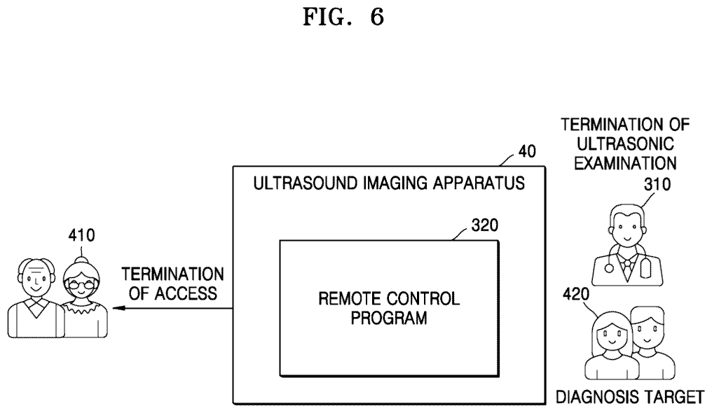

FIG. 6 is a diagram showing termination of remote access to the ultrasonic imaging apparatus 40 according to an embodiment.

The ultrasound imaging apparatus 40 may terminate an ultrasonic examination. The ultrasound imaging apparatus 40 may terminate an ongoing ultrasonic examination of the diagnosis target 420 under a control by the medical personnel 310.

The ultrasound imaging apparatus 40 may terminate the activated remote access in response to the termination of the ultrasonic examination. The ultrasonic imaging apparatus 40 may terminate the access of the remote accessor 410 without a special control process when the ultrasonic examination terminates. The ultrasonic imaging apparatus 40 may forcibly terminate the access of the remote accessor 410 when the ultrasonic examination terminates. For example, the ultrasound imaging apparatus 40 may terminate the activated remote access by revoking the remote access authority granted to the remote accessor 410 in response to the termination of the ultrasonic examination.

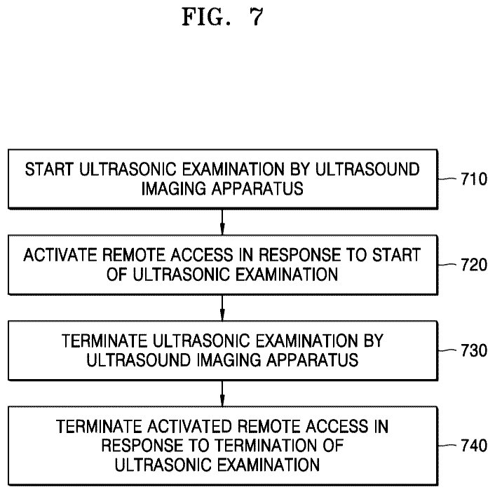

FIG. 7 is a flowchart of a method of remotely accessing the ultrasonic imaging apparatus 40 according to an embodiment.

In operation 710, the ultrasound imaging apparatus 40 may start an ultrasonic examination. The ultrasound imaging apparatus 40 may start an ultrasonic examination of a diagnosis target under a control by a medical personnel.

In operation 720, the ultrasound imaging apparatus 40 may activate remote access in response to the start of the ultrasonic examination. The ultrasound imaging apparatus 40 may transmit an access link capable of remote access to a remote accessor in response to the start of the ultrasonic examination. The remote accessor may remotely access the ultrasound imaging apparatus 40 by using the access link. The ultrasound imaging apparatus 40 may provide a limited remote access authority to the remote accessor.

In operation 730, the ultrasound imaging apparatus 40 may terminate the ultrasonic examination. The ultrasound imaging apparatus 40 may terminate an ultrasonic examination of a diagnosis target under a control by the medical personnel.

In operation 740, the ultrasound imaging apparatus 40 may terminate the activated remote access in response to the termination of the ultrasonic examination. The ultrasound imaging apparatus 40 may automatically terminate the remote access of the remote accessor in response to the termination of the ultrasonic examination.

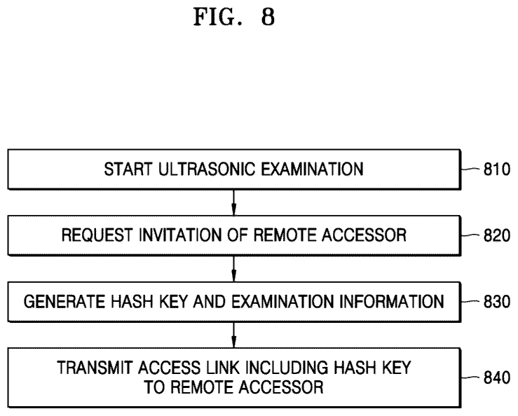

FIG. 8 is a flowchart of a method, performed by the ultrasonic imaging apparatus 40, of activating remote access, according to an embodiment.

In operation 810, the ultrasound imaging apparatus 40 may start an ultrasonic examination. The ultrasound imaging apparatus 40 may start an ultrasonic examination of a diagnosis target under a control by a medical personnel. The ultrasound imaging apparatus 40 may generate an ultrasonic examination ID for the diagnosis target when starting an ultrasound examination.

In operation 820, the ultrasound imaging apparatus 40 may request invitation of a remote accessor. The ultrasound imaging apparatus 40 may receive a command of inviting a remote accessor under a control by the medical personnel. The ultrasound imaging apparatus 40 may receive an invitation command of the medical personnel.

In operation 830, the ultrasound imaging apparatus 40 may generate a hash key and examination information. The ultrasound imaging apparatus 40 may generate the hash key, based on the value of the generated ultrasonic examination ID. The ultrasound imaging apparatus 40 may transmit a hash key generation request to the server 330 so that the server 330 generates the hash key. The ultrasound imaging apparatus 40 may generate examination information associated with the ultrasound examination. The ultrasound imaging apparatus 40 may store the generated hash key and the generated examination information.

In operation 840, the ultrasound imaging apparatus 40 may transmit an access link including the hash key to the remote accessor. For example, the ultrasound imaging apparatus 40 may transmit the URL address of the access link including the hash key to the e-mail of the remote accessor who is to be invited. For example, the ultrasound imaging apparatus 40 may transmit the access link including the hash key as a QR code to an external device of the remote accessor who is to be invited.

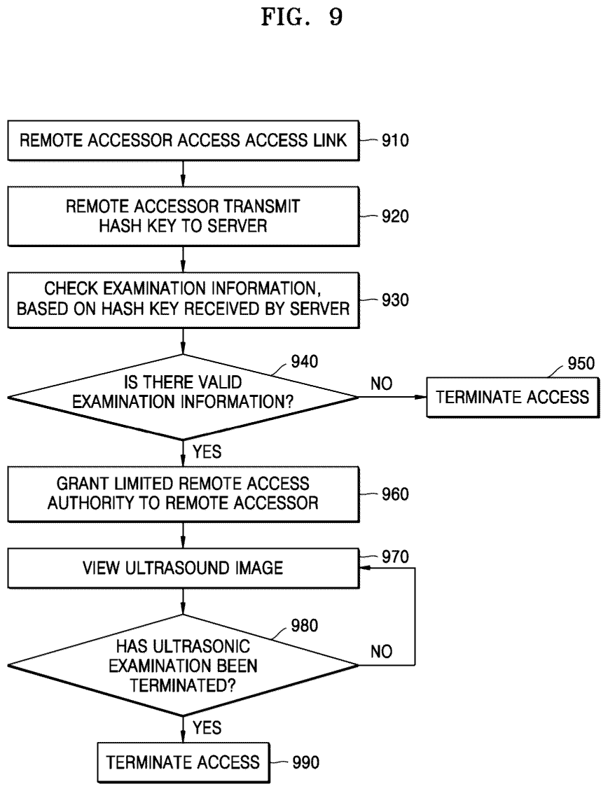

FIG. 9 is a flowchart of a method, performed by the ultrasonic imaging apparatus 40, of activating and terminating remote access, according to an embodiment.

In operation 910, a remote accessor may access an access link. The remote accessor may receive the access link transmitted by the ultrasound imaging apparatus 40. The remote accessor may access an invitation link including a hash key. The remote accessor may access the invitation link including the hash key by clicking an URL of the invitation link.

In operation 920, the remote accessor may transmit the hash key to the server 330. The server 330 may receive the hash key.

In operation 930, the server 330 may check examination information, based on the received hash key. The server 330 may search for invitation information by using the received hash key. The server 330 may authenticate whether the examination information is valid, by using the received hash key. The server 330 may determine whether there is a valid ultrasonic examination ID's value that matches with the hash key which a user has accessed. The server 330 may transmit a result of the checking of the examination information to the ultrasound imaging apparatus 40.

In operation 940, the ultrasound imaging apparatus 40 may determine whether there is valid examination information. The ultrasound imaging apparatus 40 may receive, from the server 330, a result of the determination as to whether there is a valid ultrasonic examination ID's value. The ultrasound imaging apparatus 40 may proceed to operation 960 when there is valid examination information (operation 940—YES). The ultrasound imaging apparatus 40 may proceed to operation 950 when there is no valid examination information (operation 940—NO).

In operation 950, the ultrasound imaging apparatus 40 may terminate the access. The ultrasound imaging apparatus 40 may terminate the remote access when there is no valid examination information.

In operation 960, the ultrasound imaging apparatus 40 may grant limited remote access authority to the remote accessor. The ultrasound imaging apparatus 40 may grant ultrasonic examination inquiry authority to the remote accessor. The ultrasound imaging apparatus 40 may grant the remote accessor authority to temporarily inquire an ultrasonic examination. The ultrasound imaging apparatus 40 may connect the remote accessor to an ultrasonic examination streaming room.

In operation 970, the remote accessor may view an ultrasound image. The remote accessor may view the ultrasound image obtained by the ultrasound imaging apparatus 40. The ultrasonic imaging apparatus 40 may display the ultrasound image in real time on an external device of the remote accessor so that the remote accessor may view the ultrasound image obtained by the ultrasound imaging apparatus 40, based on the limited remote access authority. The remote accessor may watch an ultrasonic examination progress.

In operation 980, the ultrasound imaging apparatus 40 may determine whether an ultrasonic examination has been terminated. The ultrasound imaging apparatus 40 may proceed to operation 990 when an ultrasonic examination has been terminated (operation 980—YES). The ultrasound imaging apparatus 40 may return to operation 970 so that the remote accessor may viewthe ultrasound image, when an ultrasonic examination has not been terminated (operation 980—NO).

In operation 990, the ultrasound imaging apparatus 40 may terminate the access. The ultrasound imaging apparatus 40 may terminate the remote access authority granted to the remote accessor, when an ultrasonic examination has been terminated. The ultrasound imaging apparatus 40 may automatically terminate a page displaying the ultrasound image shared by the remote accessor, when an ultrasonic examination has been terminated.

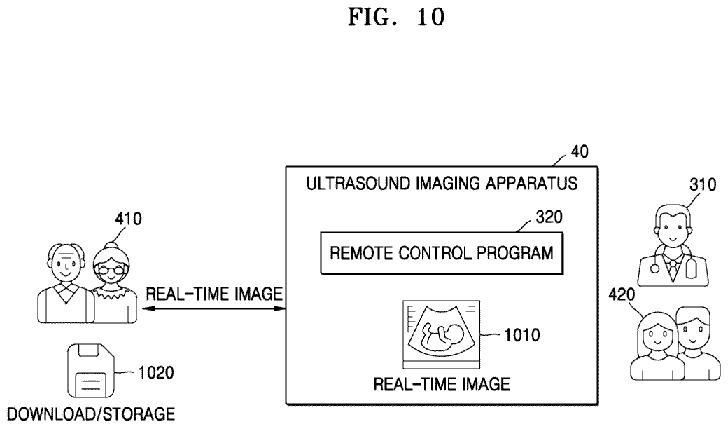

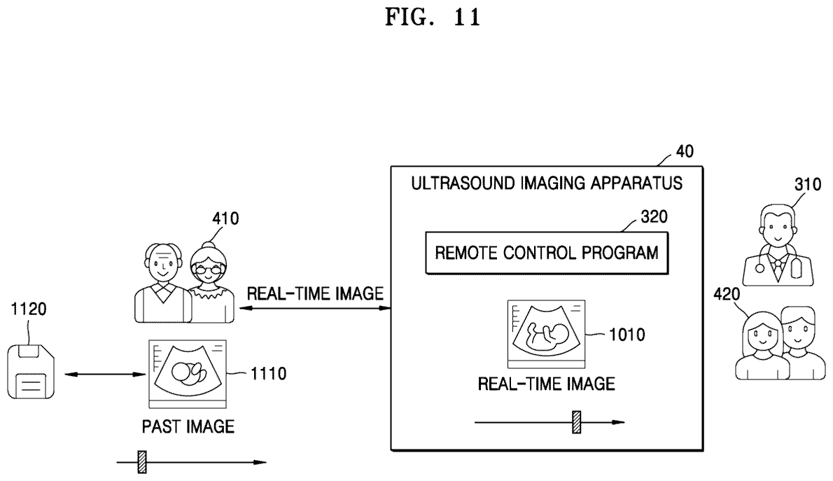

FIG. 10 is a diagram showing storage 1020 of a real-time image 1010 during remote access to the ultrasonic imaging apparatus 40 according to an embodiment.

The ultrasound imaging apparatus 40 may remotely access the remote accessor 410. The ultrasound imaging apparatus 40 may obtain an ultrasound image of the diagnosis target 420 under a control by the medical personnel 310. The ultrasound imaging apparatus 40 may generate the real-time image 1010 by obtaining the ultrasound image in real time. The ultrasound imaging apparatus 40 may provide the real-time image 1010 to the remote accessor 410.

The ultrasound imaging apparatus 40 may grant real-time image storage authority to the remote accessor 410 so that the remote accessor 410 may download or store the real-time image 1010 displayed in real time. The remote accessor 410 may download the received real-time image 1010 from the ultrasound diagnosis apparatus 40. The remote accessor 410 may store the downloaded real-time image 1010, as indicated by 1020. The ultrasound imaging apparatus 40 may grant real-time image storage authority to the remote accessor 420 so that the remote accessor 410 may download or store the ultrasound image of the diagnosis target 420 in real time at a desired time point.

FIG. 10 is a diagram showing storage 1120 of a past image 1110 during remote access to the ultrasonic imaging apparatus 40 according to an embodiment.

The ultrasound imaging apparatus 40 may remotely access the remote accessor 410. The ultrasound imaging apparatus 40 may obtain an ultrasound image of the diagnosis target 420 under a control by the medical personnel 310. The ultrasound imaging apparatus 40 may generate the real-time image 1010 by obtaining the ultrasound image in real time. The ultrasound imaging apparatus 40 may provide the real-time image 1010 to the remote accessor 410.

The ultrasound imaging apparatus 40 may grant past image storage authority to the remote accessor 410 so that the remote accessor 410 may download or store the past image 1110 displayed after an ultrasonic examination starts. The remote accessor 410 may go back to a past portion of the real-time image 1010 and check the past image 1110. The remote accessor 410 may download the past image 1110. The remote accessor 410 may store the downloaded past image 1110. The ultrasound imaging apparatus 40 may grant a past image storage authority to the remote accessor 420 so that the remote accessor 410 may download or store an ultrasound image of a desired past specific point of time from among the diagnosis target 420.

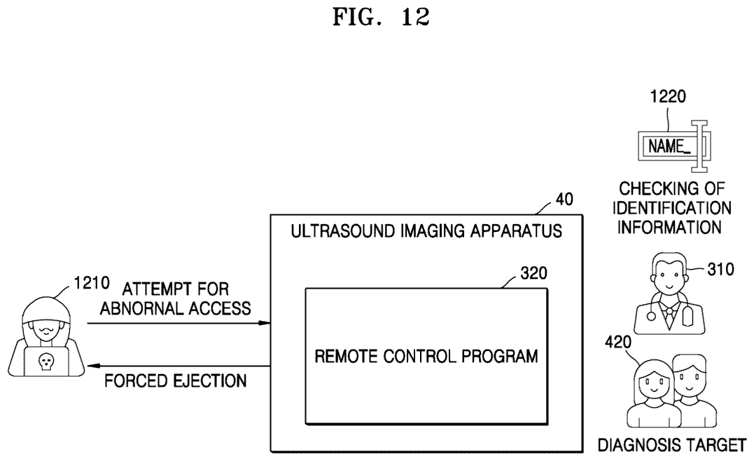

FIG. 12 is a diagram showing blocking of abnormal access during remote access to the ultrasonic imaging apparatus 40 according to an embodiment.

The ultrasound imaging apparatus 40 may obtain an ultrasound image of the diagnosis target 420 under a control by the medical personnel 310.

An abnormal accessor 1210, who is an external, unspecified user, may attempt an abnormal remote access to the ultrasound imaging apparatus 40. When the abnormal accessor 1210 attempts an abnormal access, the ultrasonic imaging apparatus 40 may check identification information 1220. For example, the ultrasound imaging apparatus 40 may check an access name or access ID of the abnormal accessor 1210.

The ultrasonic imaging apparatus 40 may forcibly eject the abnormal accessor 1210 in response to detection of the abnormal access attempted by the abnormal accessor 1210. The ultrasound imaging apparatus 40 may take forced exit measures when the abnormal accessor 1210 remotely accesses the ultrasound imaging apparatus 40.

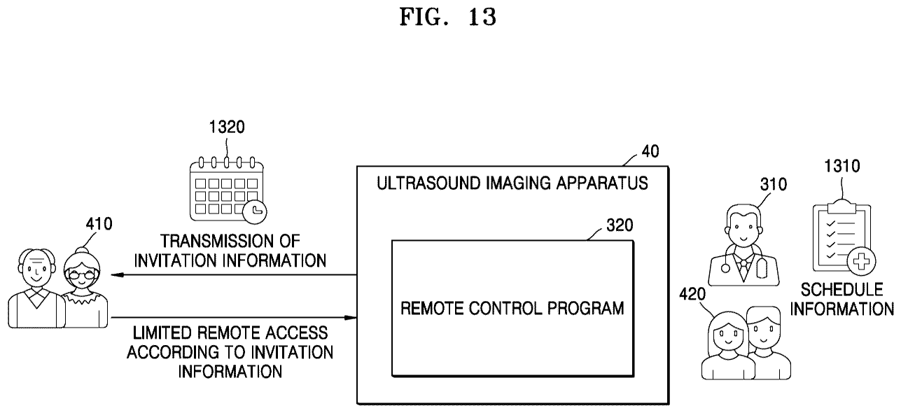

FIG. 13 is a diagram showing transmission of the invitation information 1320 and provision of a remote access authority by the ultrasonic imaging apparatus 40, according to an embodiment.

The ultrasound imaging apparatus 40 may obtain an ultrasound image of the diagnosis target 420 under a control by the medical personnel 310.

The ultrasonic imaging apparatus 40 may transmit the invitation information 1320 including schedule information 1310 to the remote accessor 410. The schedule information 1310 may be information related to the schedule of an ultrasound examination of the diagnosis target 420. For example, the schedule information 1310 may be an ultrasonic examination schedule of the diagnosis target 420. The ultrasonic imaging apparatus 40 may transmit the invitation information 1320 to the remote accessor 410 earlier than the schedule of an ultrasound examination of the diagnosis target 420 in connection with the schedule information 1310.

The ultrasound imaging apparatus 40 may provide limited remote access authority to the remote accessor 410, based on the invitation information 1320. The ultrasound imaging apparatus 40 may transmit, to the remote accessor 410, a notification informing that the invitation information 1320 has been transmitted. The remote accessor 410 may ascertain from the notification that the invitation information 1320 has been received. The remote accessor 410 may remotely access the ultrasound imaging apparatus 40, based on the invitation information 1320. The remote accessor 410 may remotely access the ultrasound imaging apparatus 40 at a date and time matching the schedule information 1310 included in the invitation information 1320, and view the ultrasound image.

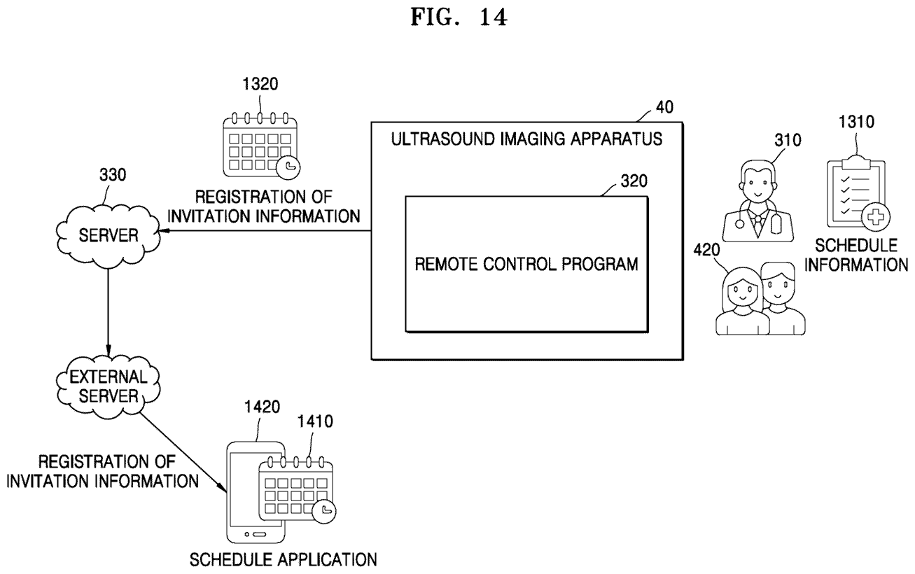

FIG. 14 is a diagram showing registration of the invitation information 1320 and provision of a remote access authority by the ultrasonic imaging apparatus 40, according to an embodiment.

The ultrasound imaging apparatus 40 may obtain an ultrasound image of the diagnosis target 420 under a control by the medical personnel 310.

The ultrasonic imaging apparatus 40 may transmit the invitation information 1320 including the schedule information 1310 to the server 330. The schedule information 1310 may be information related to the schedule of an ultrasound examination of the diagnosis target 420. For example, the schedule information 1310 may be an ultrasonic examination schedule of the diagnosis target 420. The ultrasonic imaging apparatus 40 may transmit the invitation information 1320 to the server 330 earlier than the schedule of an ultrasound examination of the diagnosis target 420 in connection with the schedule information 1310. The server 330 may previously register the schedule information 1310 in an external server of another company. For example, when an ultrasound examination schedule is reserved according to the schedule information 1310, the server 330 may register the schedule information 1310 in a linked calendar system server according to authentication information of the diagnosis target 420.

The ultrasonic imaging apparatus 40 may register the invitation information 1320 in a schedule application 1410 of a remote accessor though the server 330. The invitation information 1320 registered in the schedule application 1410 of the remote accessor may be confirmed through an external device 1420 of the remote accessor. The remote accessor may check the invitation information 1320 through the schedule application 1410. The remote accessor may remotely access the ultrasound imaging apparatus 40, based on the invitation information 1320. The remote accessor 410 may remotely access the ultrasound imaging apparatus 40 at a date and time matching the schedule information 1310 included in the invitation information 1320, and view the ultrasound image.

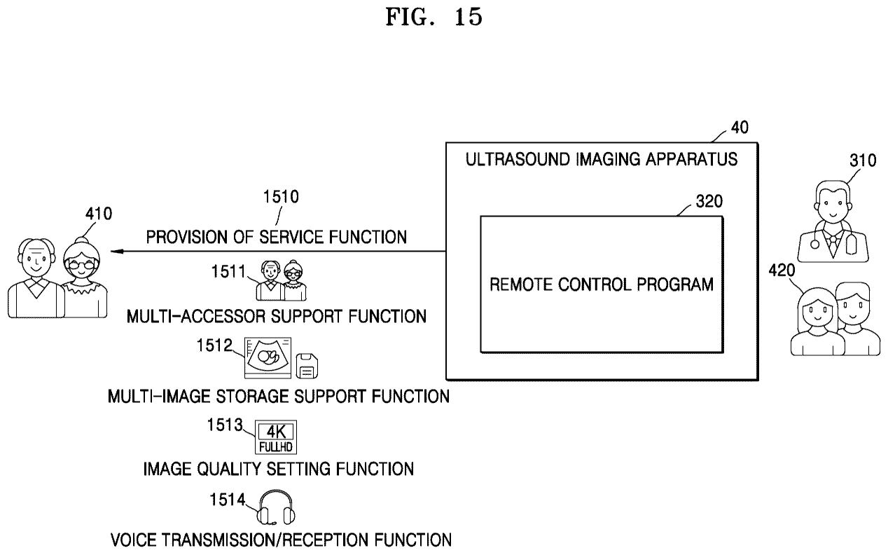

FIG. 15 is a diagram showing provision of a service function 1510 during remote access to the ultrasonic imaging apparatus 40 according to an embodiment.

The ultrasound imaging apparatus 40 may remotely access the remote accessor 410. The ultrasound imaging apparatus 40 may obtain an ultrasound image of the diagnosis target 420 under a control by the medical personnel 310. The ultrasound imaging apparatus 40 may provide the ultrasound image to the remote accessor 410.

The ultrasound imaging apparatus 40 may provide at least one service function 1510 related to the remote access to the remote accessor 410. The at least one service function 1510 may include at least one of a multi-accessor support function 1511, a multi-image storage support function 1512, an image quality setting function 1513, and a voice transmission/reception function 1514. The multi-accessor support function 1511 may be a function of supporting a plurality of remote accessors 410 to simultaneously remotely access the ultrasound imaging apparatus 40. The multi-image storage support function 1512 may be a function of supporting the remote accessor 410 to store the real-time image 1010 and the past image 1110 as images as many times as desired. The image quality setting function 1513 may be a function of setting the resolution of the ultrasound image shared by the remote accessor 410 to a high resolution (e.g., 4K Full HD). The voice transmission/reception function 1514 may be a function of sharing even the voice of the ultrasound image with the remote accessor 410.

The ultrasound imaging apparatus 40 may limitedly provide the at least one service function 1510 to the remote accessor 410. The ultrasound imaging apparatus 40 may selectively provide the at least one service function 1510 to the remote accessor 410 according to whether the remote accessor 410 desires the provision.

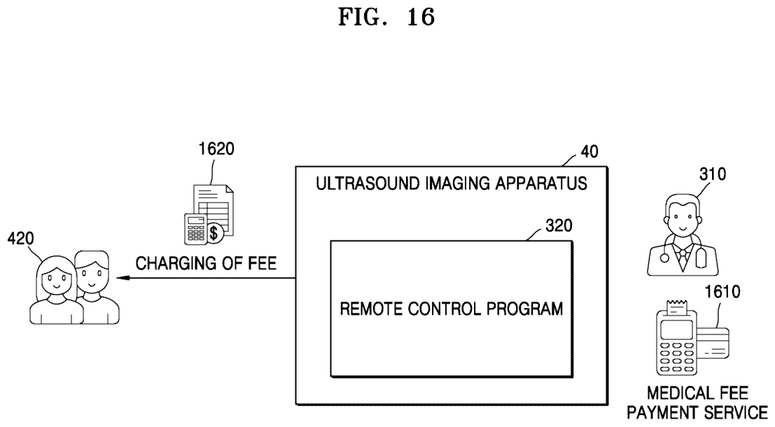

FIG. 16 is a diagram showing charging of a fee 1620 for the service function 1510 provided during remote access to the ultrasonic imaging apparatus 40 according to an embodiment.

The ultrasound imaging apparatus 40 may conduct an ultrasonic examination of the diagnosis target 420 under a control by the medical personnel 310. After the ultrasound examination is completed, the medical personnel 310 may charge the diagnosis target 420 for the ultrasound examination by using a medical fee payment service 1610 related to the ultrasound examination.

The ultrasound imaging apparatus 40 may charge the fee 1620 for the service function 1510 provided during the remote access to the ultrasound imaging apparatus 40, based on the medical fec payment service 1610 related to the ultrasound examination. For example, when the remote accessor 410 uses the image quality setting function 1513, the ultrasound imaging apparatus 40 may charge a fee 1620 for using the image quality setting function 1513 in addition to the medical fee charged using the medical fee payment service 1610.

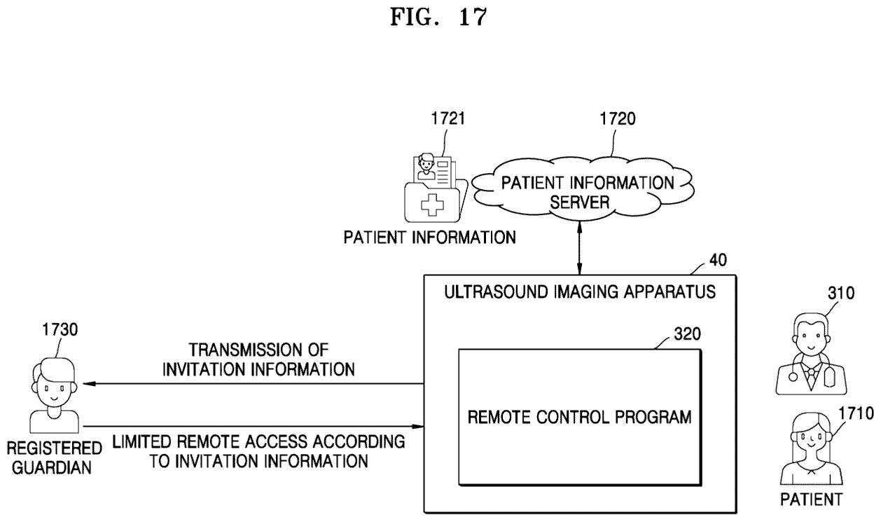

FIG. 17 is a diagram showing transmission of invitation information based on patient information 1721, by the ultrasonic imaging apparatus 40, and provision of remote access, according to an embodiment.

The ultrasound imaging apparatus 40 may obtain an ultrasound image of a patient 1710 under a control by the medical personnel 310.

The image processing apparatus 40 may obtain the patient information 1721 from a patient information server 1720. The patient information 1721 may include information about a registered guardian 1730 registered as an official guardian of the patient 1710. The image processing apparatus 40 may identify the registered guardian 1730, based on the obtained patient information 1721. The ultrasound imaging apparatus 40 may transmit the invitation information to the identified registered guardian 1730. The image processing apparatus 40 may invite the registered guardian 1730 registered in the patient information 1721 when the ultrasound examination starts in connection with a patient information system.

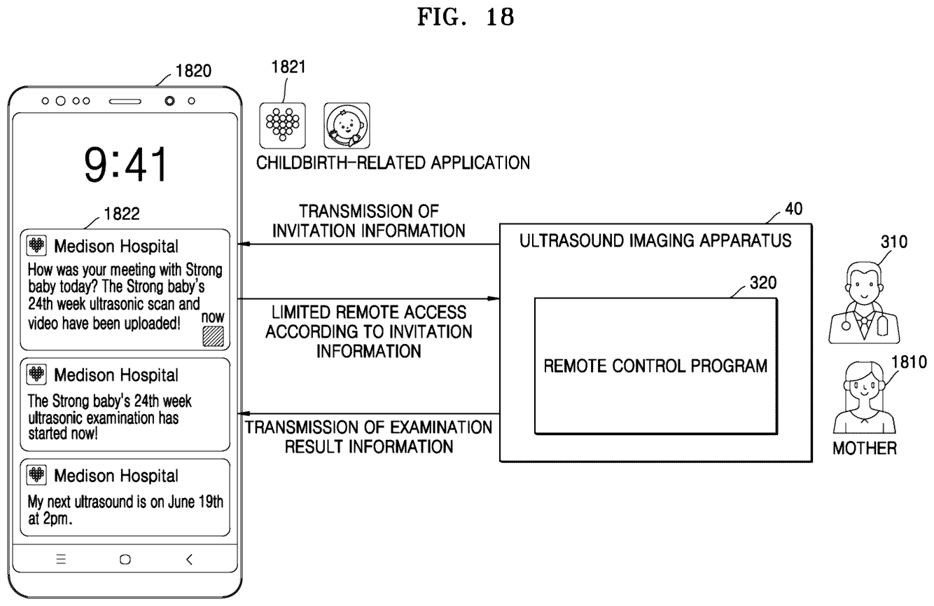

FIG. 18 is a diagram showing transmission of invitation information to an external device 1820, provision of a remote access authority, and transmission of examination result information, by the ultrasonic imaging apparatus 40 through an application 1821, according to an embodiment.

The ultrasound imaging apparatus 40 may obtain an ultrasound image of a mother 1810 under a control by the medical personnel 310. For example, the ultrasound imaging apparatus 40 may obtain an ultrasound image of a fetus under a control by the medical personnel 310.

The ultrasonic imaging apparatus 40 may transmit the invitation information through the application 1821 of the external device 1820 linked with the ultrasonic imaging apparatus 40. For example, the ultrasound imaging apparatus 40 may transmit the invitation information to a childbirth-related application (e.g., a maternal handbook) stored in the external device 1820.

The ultrasound imaging apparatus 40 may grant limited remote access authority to the external device 1820, based on the invitation information. The remote accessor 410 may remotely access the ultrasound imaging apparatus 40 through the application 1821 of the external device 1820.

The ultrasonic imaging apparatus 40 may transmit the examination result information 1822 related to the ultrasonic examination to the external device 1820 so that the external device 1820 may check the examination result information through the application 1821. The examination result information 1822 may include a summary of the ultrasonic examination after completion of the ultrasonic examination, an ultrasound image related to the ultrasonic examination, an image of the ultrasound image, and a comment related to the ultrasonic examination. The ultrasonic imaging apparatus 40 may transmit the examination result information 1822 to the external device 1820 after the ultrasonic examination is completed. The remote accessor 410 may check the examination result information 1822 through the application 1821.

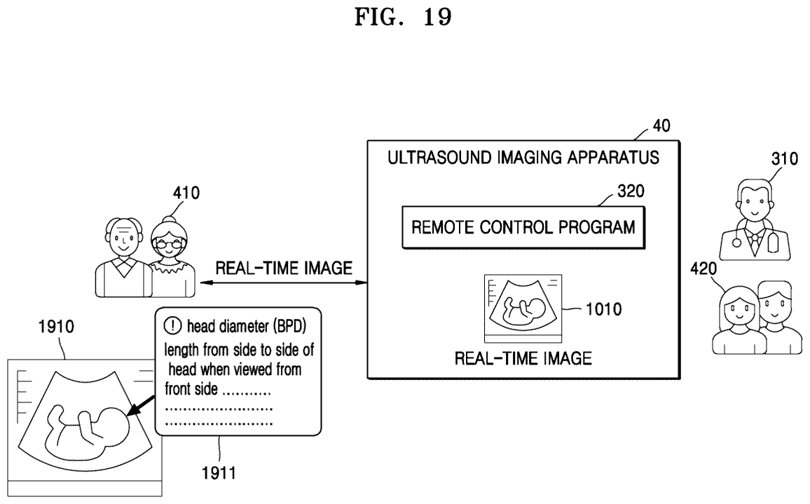

FIG. 19 is a diagram showing provision of medical information 1911 during remote access to the ultrasonic imaging apparatus 40 according to an embodiment.

The ultrasound imaging apparatus 40 may remotely access the remote accessor 410. The ultrasound imaging apparatus 40 may obtain an ultrasound image of the diagnosis target 420 under a control by the medical personnel 310. The ultrasound imaging apparatus 40 may generate the real-time image 1010 by obtaining the ultrasound image in real time. The ultrasound imaging apparatus 40 may provide the real-time image 1010 to the remote accessor 410.

The ultrasonic imaging apparatus 40 may provide a medical information display image 1910 to the remote accessor 410 to display an ultrasound image obtained by the ultrasonic imaging apparatus and the medical information 1911 related to the ultrasound image. The medical information 1911 may include information representing a result of interpreting parts of the ultrasound image that are difficult for non-medical personnel to easily identify. For example, the ultrasound imaging apparatus 40 may provide the medical information display image 1910 to the remote accessor 410 to display the medical information 1911 representing that a head diameter (BPD) of a fetus is a length from side to side of the head of the fetus when viewed from the front side while pointing to the head of the fetus, while displaying an ultrasound image of the fetus.

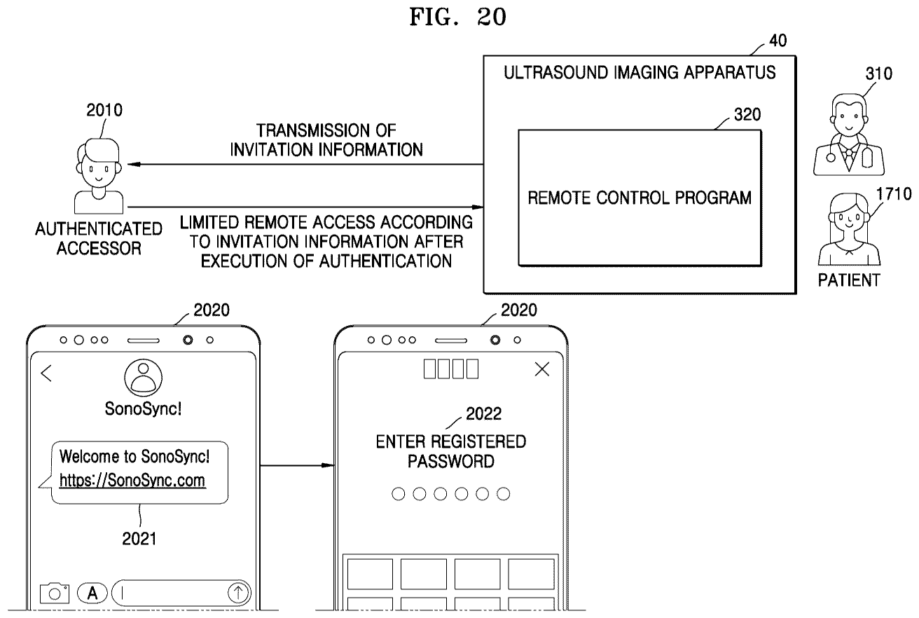

FIG. 20 is a diagram showing provision of a remote access authority by the ultrasonic imaging apparatus 40 after execution of authentication, according to an embodiment.

The ultrasound imaging apparatus 40 may obtain an ultrasound image of a patient 1710 under a control by the medical personnel 310.

The ultrasound imaging apparatus 40 may transmit invitation information including an authentication request 2021 to a remote accessor. The ultrasonic imaging apparatus 40 may transmit the invitation information including an authentication request 2021 as a text message or SNS message to an external device 2020 of the remote accessor.

The remote accessor may receive the invitation information including an authentication request 2021 through the external device 2020. The remote accessor may perform an authentication procedure 2022 in response to an authentication request 2021. For example, the remote accessor may perform authentication, such as mobile phone authentication, using the external device 2020.

The ultrasound imaging apparatus 40 may provide limited remote access authority based on the invitation information to an authenticated accessor 2010 who has completed an authentication procedure 2022 for the authentication request 2021. The ultrasonic imaging apparatus 40 may allow only remote access by the authenticated accessor 2010 who has completed the authentication procedure 2022.

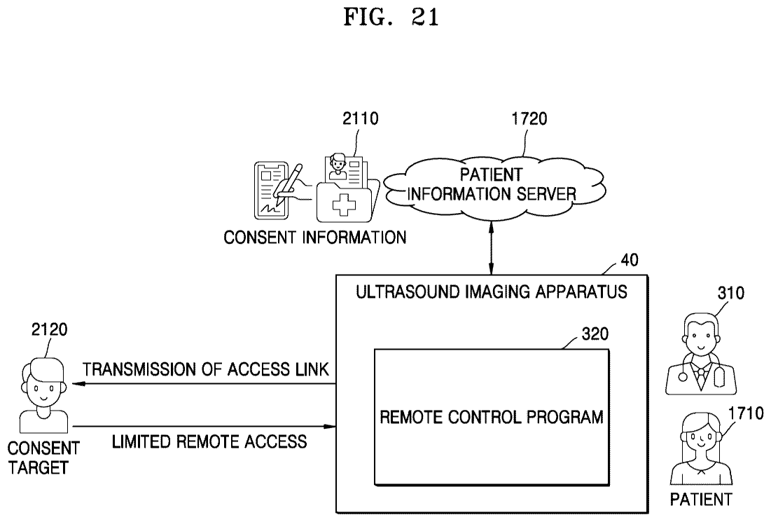

FIG. 21 is a diagram showing provision of remote access based on consent information 2110 by the ultrasonic imaging apparatus 40, according to an embodiment.

The ultrasound imaging apparatus 40 may obtain the ultrasound image of the patient 1710 under a control by the medical personnel 310.

The image processing apparatus 40 may obtain, from a patient information server 1720, the consent information 2110 indicating that the patient 1710 has approved providing information related to an ultrasound examination. For example, the consent information 2110 may include electronic signature information or authentication information of the patient 1710 that indicates that provision, through remote access, of an ultrasound image related to the ultrasound examination to a consent target 2120 registered by the patient 1710 is approved.

The ultrasound imaging apparatus 40 may transmit an access link to the consent target 2120 approved by the patient 1710, based on the consent information 2110. The ultrasound imaging apparatus 40 may provide a limited remote access authority to the consent target 2120, based on the access link. The consent target 2120 may remotely access the ultrasound imaging apparatus 40 through the access link.

The disclosure provides a method of inviting a remote accessor as a temporary guest on a one-time basis when performing an ultrasound remote diagnosis by using an ultrasound imaging apparatus.

A method of remotely accessing an ultrasound imaging apparatus according to an embodiment may include starting an ultrasonic examination by the ultrasound imaging apparatus, activating remote access in response to the starting of the ultrasonic examination, terminating the ultrasonic examination by the ultrasound imaging apparatus, and terminating the activated remote access in response to the terminating of the ultrasonic examination.

The activating of the remote access according to an embodiment may include transmitting, to a remote accessor, an access link that enables remote access to the ultrasound imaging apparatus.

The transmitting of the access link to the remote accessor according to an embodiment may include transmitting an invitation request in an email or QR code form to an external device of the remote accessor.

The transmitting of the access link to the remote accessor according to an embodiment may include generating a hash key and examination information related to the ultrasonic examination, and transmitting the access link including the hash key to the remote accessor.

The activating of the remote access according to an embodiment may include granting limited remote access authority to the remote accessor, and displaying, in real time, an ultrasound image obtained by the ultrasound imaging apparatus on an external device of the remote accessor so that the remote accessor may view the ultrasound image, based on the limited remote access authority.

The activating of the remote access according to an embodiment may include granting real-time image storage authority to a remote accessor so that the remote accessor may download or store a real-time image displayed in real time.

The activating of the remote access according to an embodiment may include granting past image storage authority to the remote accessor so that the remote accessor may download or store a past image displayed after the ultrasonic examination starts.

The method of remotely accessing the ultrasound imaging apparatus according to an embodiment may further include forcibly ejecting an abnormal accessor in response to detection of an abnormal access attempt by the abnormal accessor

The activating of the remote access according to an embodiment may include transmitting invitation information including schedule information to the remote accessor, and providing limited remote access authority to the remote accessor, based on the invitation information.

The transmitting of the invitation information to the remote accessor according to an embodiment may include registering the invitation information in a schedule application of the remote accessor through a server.

The activating of the remote access according to an embodiment may include providing at least one service function related to the remote access to the remote accessor, and the at least one service function may include at least one of a multi-accessor support function, a multi-image storage support function, an image quality setting function, or a voice transmission/reception function.

The providing of the at least one service function according to an embodiment may include charging a fee, based on a medical fee payment service related to the ultrasonic examination.

The activating of the remote access according to an embodiment may include obtaining patient information from a patient information server, identifying a registered guardian, based on the obtained patient information, and transmitting invitation information to the identified registered guardian.

The activating of the remote access according to an embodiment may include transmitting invitation information through an application of an external device linked to the ultrasound imaging apparatus, granting limited remote access authority to the external device, based on the invitation information, and transmitting examination result information related to the ultrasonic examination to the external device.

The activating of the remote access according to an embodiment may include transmitting invitation information through an application of an external device linked to the ultrasound imaging apparatus, granting limited remote access authority to the external device, based on the invitation information, and transmitting examination result information related to the ultrasonic examination to the external device so that the external device may check the examination result information.

The activating of the remote access according to an embodiment may include providing a medical information display image to the remote accessor so as to display medical information related to an ultrasound image obtained by the ultrasound imaging apparatus together with the ultrasound image.

The activating of the remote access according to an embodiment may include transmitting invitation information including an authentication request to the remote accessor, and providing a limited remote access authority based on the invitation information to an authenticated accessor who has completed an authentication procedure for the authentication request.

The activating of the remote access according to an embodiment may include obtaining, from a patient information server, consent information indicating that a patient has approved providing information related to the ultrasound examination, transmitting an access link to a consent target approved by the patient, based on the consent information, and providing a limited remote access authority to the consent target, based on the access link.

A method of remotely accessing an ultrasound imaging apparatus according to an embodiment may include starting an ultrasonic examination by the ultrasound imaging apparatus, and generating remote access path for activating remote access, in response to the starting of the ultrasonic examination.

A method of remotely accessing an ultrasound imaging apparatus according to an embodiment may include terminating an ultrasonic examination currently being conducted, by the ultrasound imaging apparatus, and terminating the activated remote access by revoking remote access authority granted to a remote accessor, in response to the terminating of the ultrasonic examination.

In a method of remote access to an ultrasound imaging apparatus according to the disclosure, remote access is activated when an ultrasonic examination starts and the remote access is terminated when the ultrasonic examination ends, in connection with the ultrasonic examination, whereby the inconvenience of a medical personnel using the ultrasound imaging device having to directly provide remote access to a remote accessor and terminate the remote access may be addressed.

A limited remote access authority is provided to a remote accessor of an ultrasound imaging apparatus according to the disclosure, whereby the remote accessor may be prevented from interfering with a medical process with many unnecessary authorities.

In the method of remote access to an ultrasonic imaging apparatus according to the disclosure, the remote accessor may conveniently access the ultrasonic imaging apparatus remotely by granting access authority through an access link.

An apparatus, method, or computer program according to an embodiment perform an operation related to artificial intelligence (AI). An operation related to AI is executed through a processor and a memory. The processor may perform the operation related to AI by using one processor or a plurality of processors. The one or plurality of processors may be a general-purpose processor such as a central processing unit (CPU), an application processor (AP), or a digital signal processor (DSP), a graphics-only processor such as a graphics processing unit (GPU) or a vision processing unit (VPU), or an artificial intelligence (AI)-only processor such as a neural processing unit (NPU). The one or plurality of processors process input data, according to a program, instruction, or AI model stored in memory.

The program, instruction, or AI model related to AI may be created through machine learning. Here, being created through learning means that a basic AI model is trained using a plurality of training data by a learning algorithm, so that a program, instruction, or AI model performing desired characteristics (or a desired purpose) is created. Such learning may be performed in a device itself on which an AI operation according to an embodiment is performed, or may be performed through a separate server and/or system. Examples of the learning algorithm include, but are not limited to, supervised learning, unsupervised learning, semi-supervised learning, or reinforcement learning.

The AI model may be composed of a plurality of neural network layers. Each of the plurality of neural network layers has a plurality of weight values, and performs a neural network operation through an operation between an operation result of a previous layer and the plurality of weight values. The plurality of weight values of the plurality of neural network layers may be optimized by a learning result of the AI model. An artificial neural network may include a deep neural network (DNN), for example, a Convolutional Neural Network (CNN), a Deep Neural Network (DNN), a Recurrent Neural Network (RNN), a Restricted Boltzmann Machine (RBM), a Deep Belief Network (DBN), a Bidirectional Recurrent Deep Neural Network (BRDNN), or a Deep Q-Networks, but embodiments of the disclosure are not limited thereto.

The machine-readable storage medium may be provided as a non-transitory storage medium. The ‘non-transitory storage medium’ is a tangible device and only means that it does not contain a signal (e.g., electromagnetic waves). This term does not distinguish a case in which data is stored semi-permanently in a storage medium from a case in which data is temporarily stored. For example, the non-transitory recording medium may include a buffer in which data is temporarily stored.

According to an embodiment, methods according to embodiments may be provided by being included in a computer program product. The computer program product, which is a commodity, may be traded between sellers and buyers. Computer program products are distributed in the form of device-readable storage media (e.g., compact disc read only memory (CD-ROM)), or may be distributed (e.g., downloaded or uploaded) through an application store or between two user devices (e.g., smartphones) directly and online. In the case of online distribution, at least a portion of the computer program product (e.g., a downloadable app) may be stored at least temporarily in a device-readable storage medium, such as a memory of a manufacturer's server, a server of an application store, or a relay server, or may be temporarily generated.

What is claimed is:

1. A method of remotely accessing an ultrasound imaging apparatus, the method comprising:
starting an ultrasonic examination by the ultrasound imaging apparatus;
activating remote access in response to the starting of the ultrasonic examination;
terminating the ultrasonic examination by the ultrasound imaging apparatus; and
terminating the activated remote access in response to the terminating of the ultrasonic examination.

2. The method of claim 1, wherein the activating of the remote access comprises transmitting, to a remote accessor, an access link that enables remote access to the ultrasound imaging apparatus.

3. The method of claim 2, wherein the transmitting of the access link to the remote accessor comprises transmitting an invitation request in an email or QR code form to an external device of the remote accessor.

4. The method of claim 2, wherein the transmitting of the access link to the remote accessor comprises:
generating a hash key and examination information related to the ultrasonic examination; and
transmitting the access link including the hash key to the remote accessor.

5. The method of claim 1, wherein the activating of the remote access comprises:
granting limited remote access authority to a remote accessor; and
displaying, in real time, an ultrasound image obtained by the ultrasound imaging apparatus on an external device of the remote accessor so that the remote accessor is able to view the ultrasound image, based on the limited remote access authority.

6. The method of claim 1, wherein the activating of the remote access comprises granting real-time image storage authority to a remote accessor so that the remote accessor is able to download or store a real-time image displayed in real time.

7. The method of claim 1, wherein the activating of the remote access comprises granting past image storage authority to a remote accessor so that the remote accessor is able to download or store a past image displayed after the ultrasonic examination starts.

8. The method of claim 1, further comprising forcibly ejecting an abnormal accessor in response to detection of an abnormal access attempt by the abnormal accessor.

9. The method of claim 1, wherein the activating of the remote access comprises:
transmitting invitation information including schedule information to a remote accessor; and
providing limited remote access authority to the remote accessor, based on the invitation information.

10. The method of claim 9, wherein the transmitting of the invitation information to the remote accessor comprises registering, through a server, the invitation information in a schedule application of the remote accessor.

11. The method of claim 1, wherein
the activating of the remote access comprises providing at least one service function related to the remote access to the remote accessor, and
the at least one service function includes at least one of a multi-accessor support function, a multi-image storage support function, an image quality setting function, or a voice transmission/reception function.

12. The method of claim 11, wherein the providing of the at least one service function comprises charging a fee, based on a medical fee payment service related to the ultrasonic examination.

13. The method of claim 1, wherein the activating of the remote access comprises:
obtaining patient information from a patient information server;
identifying a registered guardian, based on the obtained patient information; and
transmitting invitation information to the identified registered guardian.

14. The method of claim 1, wherein the activating of the remote access comprises:
transmitting invitation information through an application of an external device linked to the ultrasound imaging apparatus;
granting limited remote access authority to the external device, based on the invitation information; and
transmitting examination result information related to the ultrasonic examination to the external device.

15. The method of claim 1, wherein the activating of the remote access comprises:
transmitting invitation information through an application of an external device linked to the ultrasound imaging apparatus;
granting a limited remote access authority to the external device, based on the invitation information; and
transmitting examination result information related to the ultrasonic examination to the external device so that the external device checks the examination result information.

16. The method of claim 1, wherein the activating of the remote access comprises providing a medical information display image to a remote accessor so as to display medical information related to an ultrasound image obtained by the ultrasound imaging apparatus together with the ultrasound image.

17. The method of claim 1, wherein the activating of the remote access comprises:
transmitting invitation information including an authentication request to a remote accessor; and
providing limited remote access authority based on the invitation information to an authenticated accessor who has completed an authentication procedure for the authentication request.

18. The method of claim 1, wherein the activating of the remote access comprises:
obtaining, from a patient information server, consent information indicating that a patient has approved providing information related to the ultrasound examination;
transmitting an access link to a consent target approved by the patient, based on the consent information; and
providing limited remote access authority to the consent target, based on the access link.

19. A method of remotely accessing an ultrasound imaging apparatus, the method comprising:
starting an ultrasonic examination by the ultrasound imaging apparatus; and
generating a remote access path for activating remote access, in response to the starting of the ultrasonic examination.

20. A method of remotely accessing an ultrasound imaging apparatus, the method comprising:
terminating an ultrasonic examination currently being conducted, by the ultrasound imaging apparatus; and
terminating the activated remote access by revoking remote access authority granted to a remote accessor, in response to the terminating of the ultrasonic examination.
